(19) United States
(12) Patent Application Publication
(10) Pub. No.: US 2013/0324886 A1
(43) Pub. Date: Dec. 5, 2013

(54) BLOOD SAMPLING SYRINGE

(71) Applicant: Meng-Chen Fu, Taipei (TW)

(72) Inventor: Meng-Chen Fu, Taipei (TW)

(21) Appl. No.: 13/485,897

(22) Filed: May 31, 2012

(51) Int. Cl. A61B 5/153

(52) U.S. Cl. 600/576

(57) ABSTRACT

A blood sampling syringe includes a tube body, a pressing element, a first spring, a needle holder, a second spring, and a cover. The pressing element is formed with a plurality of abutting posts for abutting against the inclined engaging portions of the base part of the needle holder. The sliding channel and the side wall formed on the inner wall of the tube body are provided to clamp or release the needle holder. The first spring and the second spring disposed in front and rear of the needle holder make the needle holder to remain in the tube body even a needle is ejected, which allows a user to operate by a smaller force more safely.

### BACKGROUND OF THE INVENTION

1. Field of the Invention

The present invention relates to a blood sampling syringe, and in particular to a blood sampling syringe, in which a needle is held by clamping means.

2. Description of Prior Art

A blood test is often performed on a blood sample extracted from a diabetic patient so as to understand the concentration of blood glucose of the diabetic patient. According to the result of the blood test, the concentration of blood glucose of the diabetic patient can be controlled properly by drugs and foods. The conventional blood sampling syringe is configured to eject a needle by a rotating action or a lateral clamping action, and the assembly of the needle holder with the blood sampling syringe is more complicated. Further, a user needs to exert a larger force to successfully eject the needle holder. Thus, the user often feels frightened or scared in operating such a conventional blood sampling syringe.

### SUMMARY OF THE INVENTION

According to one feature of the blood sampling syringe of a preferred embodiment of the invention, the inner wall of the tube body is formed with a sliding channel and a side wall.

According to one feature of the blood sampling syringe of a preferred embodiment of the invention, the ends of the pressing element are formed with an inclined abutting post respectively.

According to one feature of the blood sampling syringe of a preferred embodiment of the invention, the pressing element is formed with an accommodating portion. According to one feature of the blood sampling syringe of a preferred embodiment of the invention, one end of a base part of the needle holder is formed with a protective cap.

According to one feature of the blood sampling syringe of a preferred embodiment of the invention, the base part of the needle holder is formed to surround a needle.

According to one feature of the blood sampling syringe of a preferred embodiment of the invention, both sides of the base part of the needle holder are formed with an inclined engaging portion respectively, and one end of the inclined engaging portion is formed with a protrusion.

According to one feature of the blood sampling syringe of a preferred embodiment of the invention, one end of the base part of the needle holder is formed with a base body.

According to one feature of the blood sampling syringe of a preferred embodiment of the invention, the cover is formed with an accommodating portion.

The detailed description and technical contents of the present invention will be described in more detail with reference to preferred embodiment thereof shown in the accompanying drawings

### BRIEF DESCRIPTION OF THE DRAWINGS

FIG. 1 is a cross-sectional perspective view showing the blood sampling syringe according to the present invention;

FIG. 2 is an exploded perspective view showing the blood sampling syringe according to the present invention;

FIG. 3 is a schematic view showing an operating state of the blood sampling syringe according to the present invention;

FIG. 4 is a schematic view showing an operating state of the blood sampling syringe according to the present invention;

FIG. 5 is a schematic view showing an operating state of the blood sampling syringe according to the present invention;

FIG. 6 is a schematic view showing an operating state of the blood sampling syringe according to the present invention;

FIG. 7 is a schematic view showing an operating state of the blood sampling syringe according to the present invention;

FIG. 8 is a schematic view showing an example of a spring used in the blood sampling syringe according to the present invention;

FIG. 9 is a schematic view showing an example of a spring used in the blood sampling syringe according to the present invention; and

FIG. 10 is a schematic view showing an example of a spring used in the blood sampling syringe according to the present invention.

### DETAILED DESCRIPTION OF THE INVENTION

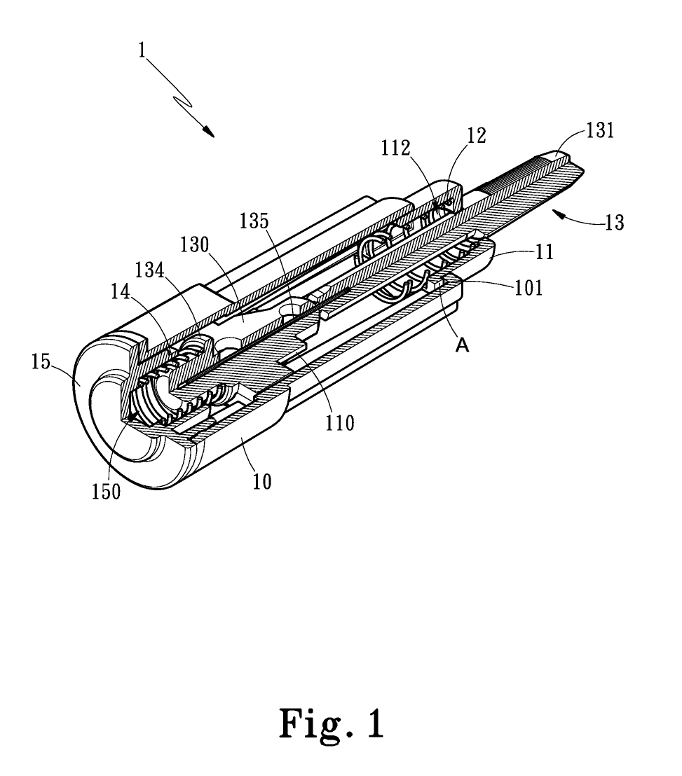

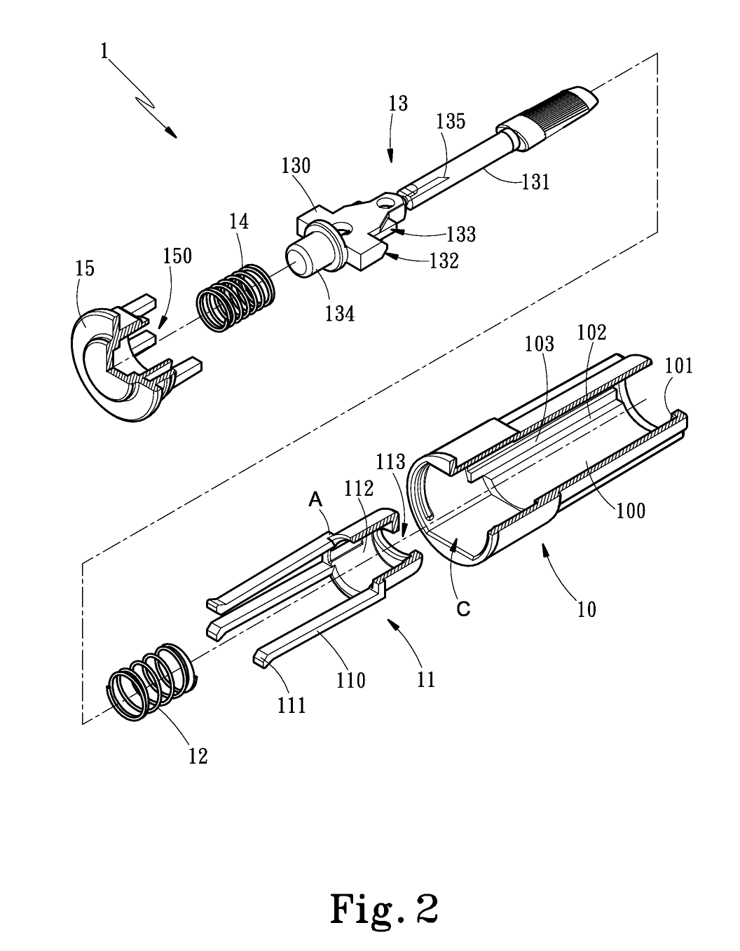

Please refer to FIGS. 1 and 2. The blood sampling syringe 1 includes: a tube body 10, a pressing element 11, a first spring 12, a needle holder 13, a second spring 14, and a cover 15. The tube body 10 is substantially formed into a hollow conical tube. Both sides of the inner wall 100 of the tube body 10 are provided with a horizontal sliding channel 102 and a vertical side wall 103. The combination of the horizontal sliding channel 102 and the vertical side wall 103 has an L-shaped cross section. Both sides of the inner wall 100 of the tube body 10 are further provided with a stopping portion 101 for abutting against the pressing element 11. After the pressing element 11 passes through the L-shaped combination of the sliding channel 102 and the side wall 103 via an end C of the tube body 10, abutting posts 110 extending from the pressing element 11 are compressed vertically by the sliding channel 102, while the abutting posts 110 are brought into tight contact with the side wall 103. In this way, inclined surfaces 111 formed on the front ends of the respective abutting posts 110 can abut against engaging portions 132 of a base part 130 of the needle holder 13, while the stopping portion 101 of the tube body 10 are abutted by an end A of the pressing element 11.

The tube body 10 allows the pressing element 11, the first spring 12, the needle holder 13, the second spring 14, and the cover 15 to be sequentially disposed therein. The pressing element 11 is formed by extending four abutting posts 110 from one end of its cap-like main body. The front end of the pressing element 11 is formed with a through-hole 113 and an accommodating portion 112. The needle holder 13 and the first spring 12 pass through the through-hole 113 to be disposed in the accommodating portion 112. The front end of the abutting post 110 is formed with an inclined surface 111 corresponding to the engaging portion 132 of the base part 130 of the needle holder 13. The pressing element 11 is disposed in the tube body 10 with the ends of the abutting posts 110 of the pressing element 11 being disposed between the sliding channel 102 and the side wall 103 of the tube body 10.

The needle holder 13 comprises a base part 130 and a protective cap 131. The center of the base part 130 is configured to surround a needle 135. Both sides of the base part 130 are formed with a right-angled protrusion 133 respectively. The right-angled protrusion 133 is configured to be abutted by the first spring 12. The rear end of the right-angled protrusion 133 extends leftward and rightward to form the inclined abutting portion 132 respectively. The abutting portions 132 are configured to be abutted by the inclined surfaces 111 of the abutting portions 110 of the pressing element 11 respectively. Further, the rear end of the base part 130 is formed with a cylinder base body 134 on which the second spring 14 is provided. By this arrangement, the needle holder 13 is disposed through the first spring 12 and the pressing element 11 to be all located in the tube body 10. The cover 15 is formed into a T shape and has a circular accommodating portion 150 on its one side for accommodating the second spring 14. The cover 14 covers the end C of the tube body 10. The first spring 12 and the second spring 14 are provided in order to eject and retract the needle holder 13. The first spring 12 and the second spring 14 are provided between the pressing element 11 and the cover 15. The blood sampling syringe 1 of the present invention is constituted by connecting the tube body 10, the pressing element 11, the first spring 12, the needle holder 13, and the second spring 14 and the cover 15. Thus, after the user takes a blood sample, the needle holder 13 remains in the tube body 10, which is safe for the user during its operation.

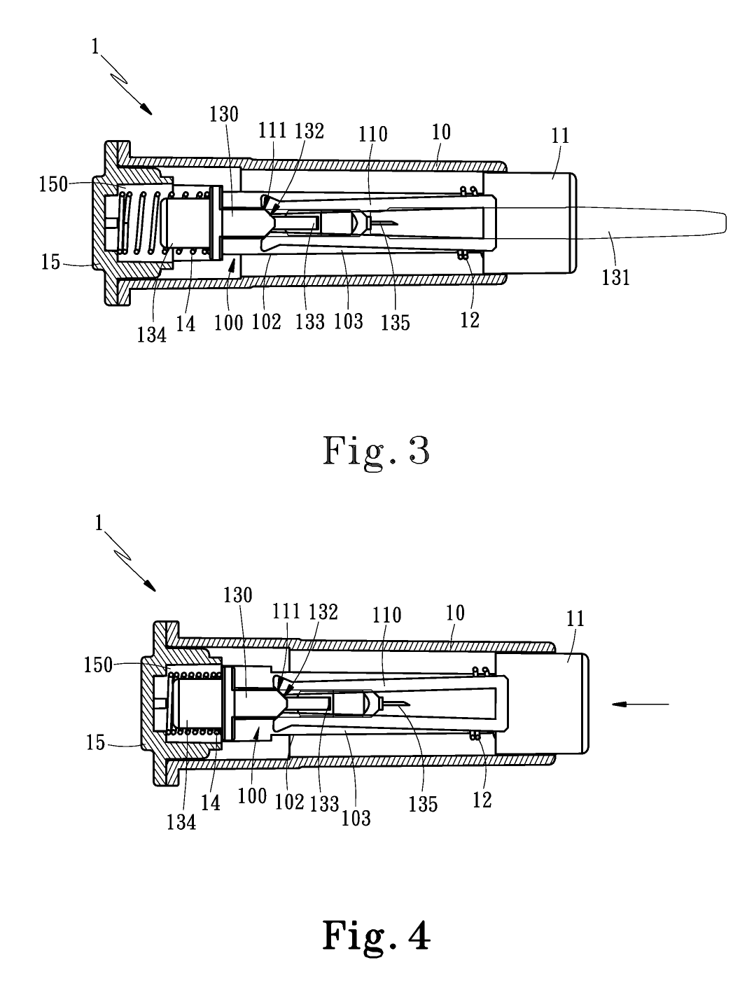

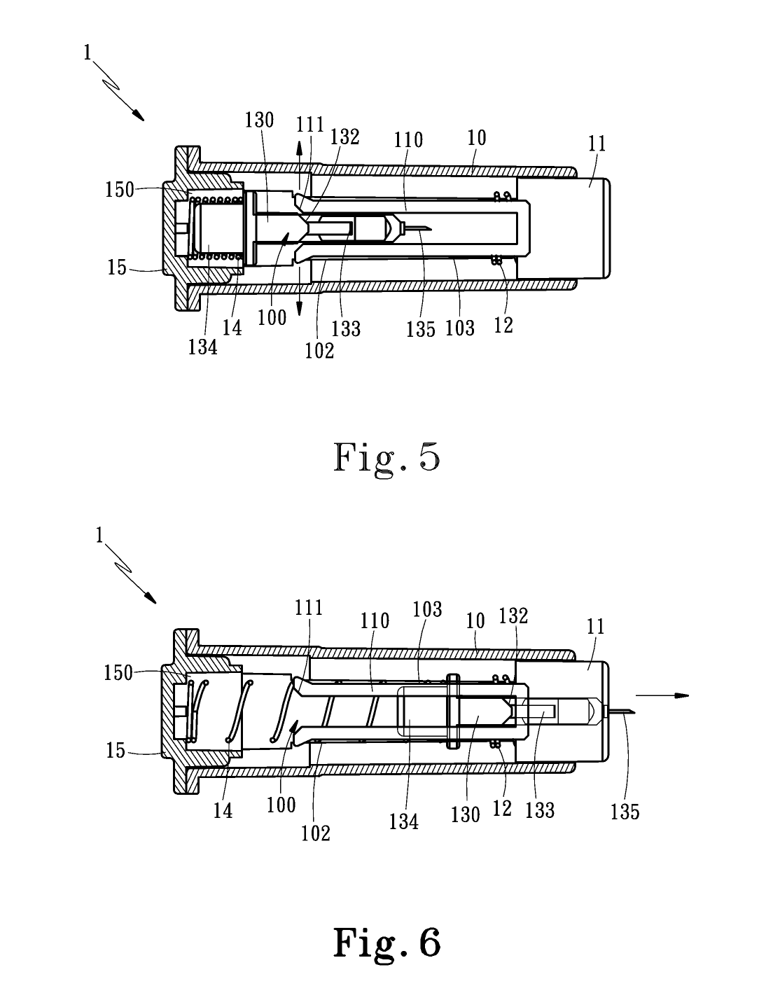

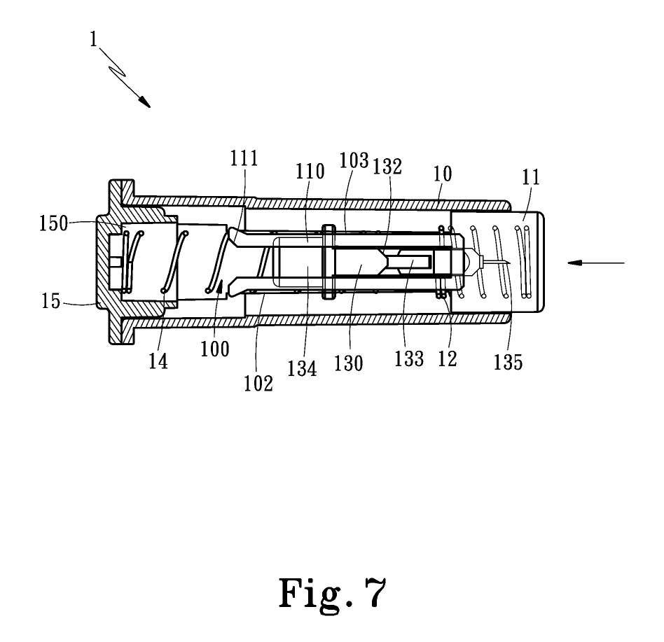

Please refer to FIGS. 3 to 7. As shown in FIG. 3, the blood sampling syringe 1 is constituted of the tube body 10, the pressing element 11, the first spring 12, the needle holder 13, the second spring 14 and the cover 15. When the pressing element 11 is disposed into the tube body 10, the abutting posts 110 of the pressing element 11 are compressed by the sliding channel 102 and the side wall 103 formed on the inner walls 100 of the tube body 10, such that the inclined surfaces 111 of the abutting posts 110 abut against the engaging portions 132 of the base part 130 of the needle holder 13. In this way, the blood sampling syringe 1 is in a state ready for use. In order to use the needle, the protective cap 131 provided on the front end of the needle holder 13 is rotatably snapped and taken out from the end of the pressing element 11.

Please refer to FIGS. 4 to 7 showing the operation of the present invention. When the user intends to take a blood sample, the user employs his/her finger pulp to press one end of the pressing element 11 toward one side. As a result, the abutting portions 110 of the pressing element 11 are compressed to slide along the sliding channel 102 and the side wall 103. When the distal ends of the abutting posts 110 of the pressing element 11 leave the sliding channel 102, the upper and lower ends of the abutting portions 110 elastically spread out to make the inclined surfaces of the abutting posts 110 leave the engaging portions 132 of the base part 130 of the needle holder 13. Since the second spring 14 provided on the rear end of the needle holder 13 is compressed to generate a larger elastic force, after the needle holder 13 leaves the abutting posts 110, the needle holder 13 is ejected forward via the second spring 14, so that the needle 135 of needle holder 13 can pass through the through-hole 113 of the pressing element 11 and then penetrate into the finger pulp of the user. At this time, the first spring 12 provided on the pressing element 11 exerts a force on the protrusions 133 formed on both sides of the base part 130 of the needle holder 13, thereby making the needle holder 13 to retract into the tube body 10. In this way, the needle holder 13 can be stayed in the tube body 10 safely after use. The elastic forces of the first spring 12 and the second spring 14 provided in front and rear of the needle holder 13 make the needle holder 13 to be maintained in the tube body 10, whereby the user can operate with a smaller force more safely.

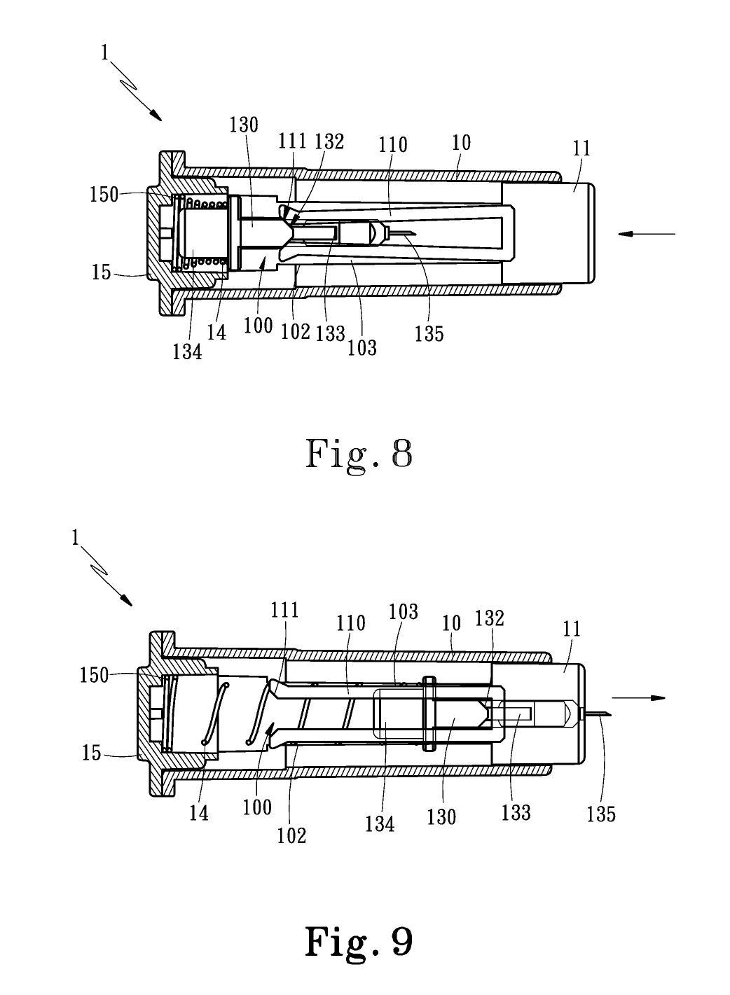

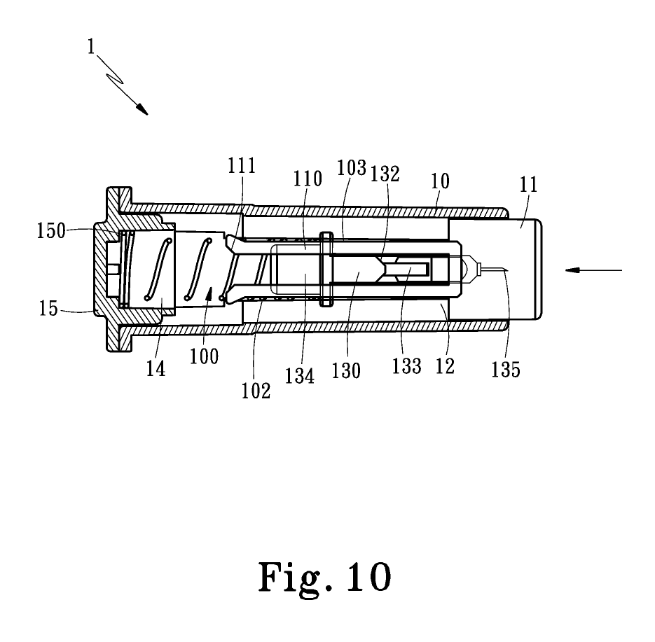

Please refer to FIGS. 8 to 10, in which only one spring is provided. The blood sampling syringe 1 is constituted of the tube body 10, the pressing element 11, the needle holder 13, the second spring 14 and the cover 15. The second spring 14 is provided between the cover 15 and the base body 134 of the needle holder 130. When the needle holder 13 is ejected to penetrate into the finger pulp of the user, the elastic restoring force of the second spring 14 pulls the needle holder 13 back into the pressing element 11 and the tube body 10. Therefore, according to the above, it can be understood that the blood sampling syringe of the present invention may be provided with one spring or two springs.

In order [bingo1] to solve the problems of the conventional blood sampling syringe and provide a novel blood sampling syringe for fast assembly and easy operation, the present Inventor provides a blood sampling syringe, which includes a tube body, a pressing element, a first spring, a needle holder, a second spring, and a cover. One end of the pressing element is formed with a plurality of abutting posts. An end surface of each abutting post is formed into an inclined surface. Both sides of a base part of the needle holder are formed with a plurality of inclined engaging portions respectively. After being assembled, the abutting posts of the pressing element abut against the inner walls of the tube body in such a manner that the inclined surfaces of the abutting posts are brought into contact with the inclined engaging portions of the needle, thereby holding the needle holder. When the pressing element is pressed inward, the abutting posts slide through a sliding channel until the abutting posts are separated from the sliding channel. In this way, the needle holder can be released. By means of the first spring and the second spring, the needle holder can be extended to penetrate a user's finger pulp or retracted back into the tube body for storage. Further, the user can operate the blood sampling syringe by a smaller force more safely.

Although the present invention has been explained in relation to its preferred embodiment, it is to be understood that many other possible modifications and variations can be made without departing from the spirit and scope of the invention as hereinafter claimed.

What is claimed is:

1. A blood sampling syringe, including:
a tube body having a sliding channel and a side wall on its inner wall;
a pressing element disposed in the tube body, the pressing element having an accommodating portion in its interior and a plurality of abutting posts extending from its one end, a distal end of each abutting post being formed with an inclined surface;
a first spring disposed in the accommodating portion of the pressing element;
a needle holder serially connecting the first spring, the pressing element and disposed in the tube body, the needle holder comprising:
a base part having symmetrical protrusions and engaging portions, an end of the base part being formed with a base body;
a protective cover located on an end of the base part; and
a needle disposed in the center of the base part;
a second spring disposed in the base body of the base part; and
a cover covering one end of the tube body, the cover having an accommodating portion corresponding to the second spring, the cover connecting the second spring, the needle holder, the first spring and the pressing element;
wherein the pressing element is pressed by an external force to make the distal ends of the abutting posts of the pressing element to slide along the sliding channel and spread out in such a manner that the inclined surfaces of the abutting posts are separated from the engaging portions of the base part of the needle holder, the needle holder is pushed outward by the second spring to make the needle extend out of one end of the tube body, and the needle holder is retracted back into the tube body via the first spring.

2. The blood sampling syringe according to claim 1, wherein one end of the tube body is formed with a stopping portion.

3. The blood sampling syringe according to claim 1, wherein one end of the pressing element abuts against the stopping portion of the tube body.

4. The blood sampling syringe according to claim 1, wherein the protrusion of the needle holder is configured to abut against the first spring.

5. The blood sampling syringe according to claim 1, wherein the engaging portions of the base part of the needle holder are configured to correspond to the inclined surfaces of the abutting posts of the pressing element.

6. A blood sampling syringe, including:
a tube body having a sliding channel and a side wall on its inner wall;
a pressing element disposed in the tube body, the pressing element having an accommodating portion in its interior and a plurality of abutting posts extending from its one end, a distal end of each abutting post being formed with an inclined surface;
a first spring disposed in the accommodating portion of the pressing element;
a needle holder serially connecting the first spring, the pressing element and disposed in the tube body, the needle holder comprising:
a base part having symmetrical protrusions and engaging portions, an end of the base part being formed with a base body;
a protective cover located on an end of the base part; and
a needle disposed in the center of the base part;
a cover covering one end of the tube body, the cover having an accommodating portion, the cover connecting a second spring, the needle holder, the first spring and the pressing element [bingo2];
wherein the pressing element is pressed by an external force to make the distal ends of the abutting posts of the pressing element to slide along the sliding channel and spread out in such a manner that the inclined surfaces of the abutting posts are separated from the engaging portions of the base part of the needle holder, the needle holder is pushed outward by the second spring to make the needle extend out of one end of the tube body, and the needle holder is retracted back into the tube body via the first spring.
